(19) United States
(12) Patent Application Publication
(10) Pub. No.: US 2009/0130196 A1
(43) Pub. Date: May 21, 2009

(54) Bacteriophage composition

(76) Inventors: Kishore Murthy, Ottawa (CA); Rainer Engelhardt, Ottawa (CA)

Correspondence Address: BRINKS, HOFER, GILSON & LIONE, P.O. BOX 1340, MORRISVILLE, NC, 27560, US

(21) Appl. No.: 11/666,704

(22) PCT Filed: Nov. 2, 2005
(86) PCT No.: PCT/CA05/01680

§ 371 Date: Nov. 19, 2008

Related U.S. Application Data
(60) Provisional application No. 60/624,576, filed on Nov. 2, 2004.
(60) Provisional application No. 60/667,589, filed on Mar. 31, 2005.

(51) Int. Cl. A61K 9/48; C12N 7/00; A61K 9/20; A23K 1/00; A61K 35/76

(52) U.S. Cl. 424/451; 435/235.1; 424/93.6; 424/464; 426/61

(57) ABSTRACT

Bacteriophage compositions, and methods for preparing bacteriophage compositions are provided. The method for producing an antibacterial composition involves adsorbing an aqueous solution of one or more bacteriophages, or one or more phage components, onto a matrix to produce a composition, and drying the composition to produce the antibacterial composition. An antibacterial composition comprising one or more strain of bacteriophage, or one or more phage component, adsorbed onto a matrix is also provided. The antibacterial composition may also be encapsulated. The antibacterial composition, or the encapsulated antibacterial composition, may be used within a cream, lotion or gel, be admixed with a pharmaceutical carrier and administered topically, orally, nasally, used as a powdered inhalant, or the antibacterial composition or encapsulated antibacterial composition, may be added to a feed for animal, aquatic or avian uses.

The present invention relates to bacteriophage compositions and their storage, preservation and use as delivery systems. More particularly, the present invention pertains to bacteriophage compositions, methods for preparing bacteriophage compositions, and uses of bacteriophage compositions as delivery systems.

### BACKGROUND OF THE INVENTION

Bacteriophage therapy has the potential to provide an effective method to specifically control the multiplication of various strains of bacteria. However, to be commercially viable, the bacteriophages themselves must show a certain degree of stability to allow for storage, for preservation and for processing into a formulation for prophylactic and therapeutic delivery.

Various methods have been used to store phage, including freezing at low temperatures, lyophilising, and storing in liquid medium. All methods have shown varying degrees of success at maintaining a high titer of viable bacteriophages.

Prouty (1953, Appl Microbiol, 1:250-351) reported that dessicated bacteriophage of lactic acid producing Streptococci remained viable at 0° C. for 42 months, at 37° C. for 72 months and at 12° C. and 25° C. for at least 78 months. However, there is no mention of the effect of storing desiccated bacteriophage on the titer of the bacteriophage.

Keogh and Pettingill (1966, Appl Microbiol, 14:4421-424) show that bacteriophages for lactic acid producing Streptococci in the presence of whey protein are resistant to freezing and cold storage. Phage stored at 4° C. and −18° C. showed little reduction in the bacteriophage titer; freeze-thaw cycles also showed no significant loss of titer. Warren and Hatch (1969, Appl Microbiol, 17:256-261) report a significant decrease in the titer and viability of a bacteriophage suspension stored (without stabilizers) at 4° C., while storage at −20° C. and 20° C. resulted in the greatest survival of phage. They also report that long term storage of bacteriophages at −20° C. tends to result in the formation of clumps.

Jepson and March (2004, Vaccine, 22:2413-2419) disclose that a liquid suspension of bacteriophages (in either SM buffer or a 1/200 dilution of SM buffer in water) was stable for 6 months at 4° C. and −70° C., with the phage remaining unaffected by freeze-thawing. Increased temperature, between 20° C. and 42° C., resulted in a significant loss of titre. Lyophilisation and immediate reconstitution of bacteriophages in the presence or absence of stabilizers resulted in a loss of titre; however lyophilization in the presence of trehalose helped reduce the damage to bacteriophages. The effect of pH of the storage medium was also examined. There was no change in bacteriophage titer over a 24 hour period at pH 3-11. However, the titer dropped rapidly when stored for 5 minutes at pH values below 2.4.

Scott et al (WO 03/093462) discloses the stabilization and immobilization of viruses, including bacteriophage, by covalently bonding the virus to a substrate. This process requires chemicals to activate the substrate and coupling agents to aid in formation of covalent bonds between the substrate and the virus. However, the virus or bacteriophages are exposed to the environment and may lose viability when subjected to hostile environment, such as low pH.

Freezing or lyophilisation of bacteriophage suspensions, or bacteriophage suspensions optionally containing stabilizers, are inconvenient methods that require specialized equipment and add to the cost of a commercial preparation. While it may be desirable to be able to store bacteriophages in a desiccated state, the process of lyophilization results in a significant loss of titre. Furthermore, the covalent attachment of bacteriophages to a substrate does not allow for the release of the bacteriophages from the substrate and may limit its usefulness for certain applications. Alternative methods for bacteriophage stabilization are required.

### SUMMARY OF THE INVENTION

The present invention relates to bacteriophage compositions and their use for storage, preservation and in delivery systems. More particularly, the present invention pertains to bacteriophage compositions, methods for preparing bacteriophage compositions, and uses of bacteriophage compositions.

It is an object of the present invention to provide a bacteriophage composition showing improved stability.

The present invention provides a method (method A) for producing bacteriophage composition comprising:

a) providing stabilized bacteriophage, phage components, or a combination thereof; and

b) encapsulating the stabilized bacteriophage, phage components, or a combination thereof, to produce the bacteriophage composition.

The present invention also pertains to the method as described above (method A), wherein the bacteriophage composition may be encapsulated using a material selected from the group consisting of vegetable fatty acids, fatty acid, stearic acid, palmitic acid, an animal wax, a vegetable wax, a wax derivative, Carnauba wax, other lipids and lipid derivatives, shellac, a polymer, a cellulose-based material, a carbohydrate-based material, or a sugar. Furthermore, the step of encapsulation (step b)) may be carried out using spinning disk atomization, fluid bed system methods for drying granulation and coating, air suspension coating, solvent evaporation, spray drying, or any other method for achieving matrix coating and encapsulation.

The present invention provides a method (method A), wherein after the step of encapsulating (step b)), the bacteriophage composition is formulated as a capsule or a tablet.

The present invention pertains to the method described above (method A), wherein in the step of providing (step a), the stabilized bacteriophage is stabilized by adsorption to a matrix. The matrix may be selected from the group consisting of skim milk powder, soya protein powder, whey protein powder, albumin powder, casein, gelatin, single cell proteins, algal protein, plant peptone, trehalose, mannitol, powdered sugar, sugar alcohol, charcoal, latex beads, a water-soluble carbohydrate-based material, talc, chitin, and fish cartilage.

The present invention pertains to the method as described above (method A), wherein, in the step of providing (step a), the stabilized bacteriophage is stabilized by adsorption to a matrix, adsorbed to a matrix and the matrix embedded in a solid support; lyophilized; lyophilized and embedded in a solid support, covalently bound to a matrix, covalently bound to a matrix and embedded in a solid support

The present invention also provides a bacteriophage composition comprising one or more than one strain of an encapsulated stabilized bacteriophage, one or more than one encapsulated phage component, one or more than one strain of a stabilized bacteriophage and one or more than one phage component encapsulated together, or a combination thereof. Furthermore, the bacteriophage composition may be formulated as a capsule or a tablet.

The present invention also pertains to a bacteriophage composition comprising one or more than one strain of a stabilized bacteriophage, one or more than one phage component, one or more than one strain of a stabilized bacteriophage and one or more than one stabilized phage component, or a combination thereof, and a pharmaceutically acceptable carrier, formulated within a tablet. The tablet may further comprise components that permit controlled release of the stabilized bacteriophage, one or more than one phage component, one or more than one strain of a stabilized bacteriophage and one or more than one stabilized phage component, or a combination thereof.

The present invention provides a bacteriophage composition one or more than one strain of a stabilized bacteriophage, one or more than one phage component, one or more than one strain of a stabilized bacteriophage and one or more than one stabilized phage component, or a combination thereof, and a pharmaceutically acceptable carrier, formulated within a capsule. The capsule may be comprised of gelatin, wax, shellac or other pharmaceutically acceptable material.

The present invention provides a method (method B) for producing an antibacterial composition comprising, embedding an aqueous solution of bacteriophages, or phage components, onto a solid or powdered matrix to produce composition, and drying the composition to produce an antibacterial composition. Further, the antibacterial composition may be encapsulated for use as a delivery system.

The present invention also pertains to the method described above (method B) wherein the matrix may be selected from the group consisting of skim milk powder, soya protein, whey protein, albumin powder, casein, gelatin, single cell proteins, trehalose, manitol, sugar and sugar alcohol, talc, chitin, fish cartilage, hydroxypropylmethylcellulose phthalate (HPMCP), cellulose acetate phthalate (CAP), or hydroxypropylmethylcellulose Acetate Succinate (HPMCAS), and the like. Furthermore, the material used to encapsulate the antibacterial composition may be selected from the group consisting of vegetable fatty acid, fatty acid, stearic acid, palmitic acid, an animal wax, a vegetable wax, Carnauba wax and other wax derivatives thereof, other lipids and lipid derivatives, shellac, a polymer, a cellulose-based material, a carbohydrate-based material, or a sugar.

The present invention also provides an antibacterial composition comprising one or more than one strain of bacteriophage, or phage component, adsorbed onto a matrix. The antibacterial composition may also be encapsulated.

The present invention includes the antibacterial material as defined above, wherein the matrix is selected from the group consisting of skim milk powder, casein, gelatin, soya protein, whey protein, albumin powder, single cell proteins, trehalose, manitol, sugar and sugar alcohol, talc, chitin, fish cartilage, and the like. Furthermore, the material used to encapsulate the antibacterial composition is selected from the group consisting of vegetable fatty acid, fatty acid, stearic acid, palmitic acid, an animal wax, a vegetable wax, Carnauba wax and other wax derivatives thereof, other lipids and lipid derivatives, shellac, a polymer, a cellulose-based material, a carbohydrate-based material, or a sugar.

The antibacterial compositions of the present invention are easy to prepare and exhibit the property of being stable over various lengths of time at refrigerator and room temperatures, from about −10° C. to about 25° C., or any amount therebetween. Furthermore, bacteriophages, or phage components, may be readily released from the antibacterial compositions of the present invention with little or no loss in titer. The antibacterial compositions of the present invention may be used within lotions, creams, gels and lubricants, toothpaste, be admixed with a pharmaceutically acceptable carrier for oral, nasal, or topical applications for example but not limited to skin, vaginal, ophthalmic, nasal, aural, anal, rectal, and other types of administration, or be used within wound dressings, and exhibit antimicrobial activity.

The antibacterial compositions of the present invention may also be encapsulated. When encapsulated, the bacteriophages, or phage components, are resistant to extended periods of exposure to low pH that would otherwise render the bacteriophages, or phage components, non-viable. Encapsulated antibacterial compositions of the present invention may be added to animal, bird or fish feed and fed to an animal, bird or fish or administered orally to humans with or without the presence of food. The encapsulation of the bacteriophages, or phage components, results in protecting the bacteriophages, or phage components, from stomach acids and increasing the duration of bacteriophage release within the gastrointestinal tract of the animal or human. It also adds stability to the phage preparation, or phage component preparation, and helps to extend its shelf life.

The present invention provides stabilized phage preparations in a dry form as a delivery system for powder inhalants. The present invention also provides a system for delivering phage or phage compositions to the gut past the stomach acids, or with appropriate formulation for controlled release of phage or phage components in the stomach.

The antibacterial compositions of the present invention may be used for human, veterinary, agricultural or aquacultural purposes. Furthermore, the compositions as described herein may be used for treatment of trees and plants, and environmental applications. The antibacterial composition, or the encapsulated antibacterial composition, may be used within a cream, lubricant, lotion or gel, be admixed with a pharmaceutical carrier and administered topically, orally, nasally, used as a powdered inhalant, or the antibacterial composition or encapsulated antibacterial composition, may be added to a feed for animal, aquatic or avian uses or administered orally to humans.

This summary of the invention does not necessarily describe all necessary features of the invention.

### BRIEF DESCRIPTION OF THE DRAWINGS

These and other features of the invention will become more apparent from the following description in which reference is made to the appended drawings wherein:

FIG. 1 shows the titer of phage applied to the skim milk powder (Before) and that obtained after immobilization and resuspension (After).

FIG. 2 shows the titer of phage applied to the soya protein powder (Before) and that obtained after immobilization and resuspension (After).

FIG. 3 shows stability of encapsulated immobilized phages over a period of 4.5 months (131 days) and 10 months (311 days) when stored at room temperature (RT) or at 4° C.

### DESCRIPTION OF PREFERRED EMBODIMENT

The present invention relates to bacteriophage compositions. More particularly, the present invention pertains to bacteriophage compositions, methods for preparing bacteriophage compositions, and uses of bacteriophage compositions.

The following description is of a preferred embodiment.

The present invention provides an antibacterial composition comprising one or more than one strain of bacteriophage, or one or more than one phage component, adsorbed onto a matrix. The present invention also provides a method for producing an antibacterial composition comprising, adsorbing an aqueous solution of bacteriophages, or phage components, onto a matrix to produce an antibacterial composition, and drying the antibacterial composition. The solution of bacteriophage, or phage components, may comprise one or more than one strain of bacteriophage, or phage component, that are capable of infecting the same or different bacterial targets. This method is simple to perform, does not require specialized equipment, and bacteriophage, or phage components, prepared in this manner are stable.

The antibacterial composition may be used in a variety of ways for the control of bacterial growth, and may be used for a variety of applications. For example, which is not to be considered limiting in any manner, the antibacterial compositions may be used for human, veterinary, agricultural and aquacultural applications including mariculture. Furthermore, the compositions may be used for treatment of trees and plants, and environmental applications. In a further non-limiting example, the antibacterial compositions of the present invention may be used within lotions, lubricants, gels and creams for dermatological or wound applications, applied directly for topical applications, for example but not limited to, applied to skin, vaginal, ophthalmic, nasal, aural, anal, or rectal areas, used within toothpaste or applied onto dental floss for oral hygiene applications. The antibacterial composition may be applied to a dressing for treating wounds. The antibacterial composition, for example an encapsulated stabilized phage, are useful as an anti-bacterial treatment for sexually transmitted disease, and may be incorporated into gels, or as condom lubricant coatings. The antibacterial composition may also be encapsulated and used as a feed additive or as an oral treatment for the control of bacteria within a human, a mammal, a fish, including finfish and shellfish species, or an avian species. For example, the phage may be formulated for delivery to certain regions of the gastrointestinal tract, for example targeting Helicobacter (cause of ulcers and stomach cancer), and the phage formulations may include acid buffers, for release in the stomach.

The term “bacteriophage” or “phage” is well known in the art and generally indicates a virus that infects bacteria. Phages are parasites that multiply inside bacterial cells by using some or all of the hosts biosynthetic machinery, and can either be lytic or lysogenic. The bacteriophages used in accordance with the present invention may be any bacteriophage, lytic or lysogenic that is effective against a target pathogen of interest.

By the term “target pathogen” or “target bacteria”, it is meant pathogenic bacteria that may cause illness in humans, animals, fish, birds, or plants. The target pathogen may be any type of bacteria, for example but not limited to bacterial species and strains of Escherichia coli, Streptococci, Humicola, Salmonella, Campylobacter, Listeria, Staphylococcus, Pasteurella, Mycobacterium, Hemophilus, Helicobacter, Mycobacterium, Mycoplasmi, Nesseria, Klebsiella, Enterobacter, Proteus, Bactercides, Pseudomonas, Borrelius, Citrobacter, Propionobacter, Treponema, Chlamydia, Trichomonas, Gonorrhea Shigella, Enterococcus, Leptospirex, Bacillii including Bacillus anthracis, Clostridium, and other bacteria pathogenic to humans, animals, fish, birds, or plants.

By the term “animal” or “animals”, it is meant any animal that may be affected by, or carry, a pathogen. For example, but without wishing to be limiting in any manner, animals may include human, poultry, such as chicken or turkey, etc; swine; livestock, which term includes all hoofed animals such a horses, cattle, goats, and sheep, etc; finfish and shellfish, and household pets such as cats and dogs.

Phage specific for one or more than one target pathogen may be isolated using standard techniques in the art for example as taught in Maniatis et al (1982, Molecular cloning: a laboratory manual, Cold Spring Harbor Laboratory, Cold Spring Harbor, N.Y.; which is incorporated herein by reference). If desired, a cocktail of different bacteriophage may be used to target one or more than one pathogen as described herein.

The term “phage component” or “phage components” refers to any phage component including but not limited to the tail, or a phage protein or other molecular assemblage that is effective in killing, reducing growth, or reproduction of a target bacteria, or a plurality of target bacteria.

If desired, a cocktail of bacteriophages, phage components, or both, may be used against a single bacterial target, or multiple bacterial tar gets. The target bacteria may be any type of bacteria, for example but not limited to the bacterial species and strains of Escherichia coli, Streptococci, Humicola, Salmonella, Campylobacter, Listeria, Lawsonia, Staphylococcus, Pasteurella, Mycobacterium, Hemophilius, Helicobacter, Mycoplasmi, Nesseria, Klebsiella, Enterobacter, Proteus, Bactercides, Pseudomonas, Borrelius, Citrobacter, Propionobacter, Treponema, Chlamydia, Trichomonas, Gonorrhea. Shigella, Enterococcus, Leptospirex, Bacillii including Bacillus anthracis, Clostridium and other bacteria pathogenic to humans, livestock, or poultry. Of interest are bacteria that are known to contaminate animal feeds, liquid animal feeds, or animal feedlots generally. Of particular interest are bacteria that also infect livestock, including swine, and poultry destined for human consumption for example but not limited to Salmonella, Campylobacter and E. coli O157:H7.

The bacteriophages, or phage components, may be provided in an aqueous solution. The aqueous solution may be any solution suitable for the purpose of the present invention. For example, the bacteriophages, or phage components, may be provided in water or in an appropriate medium as known in the art, for example LB broth, SM, TM, PBS, TBS or other common buffers known to one of skill in the art (e.g. see Maniatis et al (1981) Molecular cloning: a laboratory manual, Cold Spring Harbor Laboratory, Cold Spring Harbor, N.Y., which is incorporated herein by reference). For example, but without wishing to be limiting, the bacteriophages may be stored in LB broth.

By the term “matrix”, it is meant any suitable solid matrix that is either soluble in water, ingestible by a mammal, or suitable for use with lotions, lubricants, creams or gels. Additionally, the matrix may be non-water-soluble, provided that any absorbed phages are able to be released, either directly or indirectly (i.e. does not interfere with phage infection of bacteria), from the matrix. The matrix may be capable of adsorbing the bacteriophage, or phage components, onto its surface and releasing the bacteriophages, or phage components, either directly or indirectly, in an appropriate environment. Preferably the bacteriophages, or phage components, do not adhere so strongly to the matrix that they cannot be released upon appropriate re-suspension in a medium. For example, the adsorbed, immobilized bacteriophages, or phage components, may be non-covalently associated with the matrix so that they may be released from the matrix when desired. However, if the bacteriophage are associated with the matrix in a more substantive manner, or if the bacteriophage are covalently attached to the matrix, for example using the method of WO 03/093462 (which is incorporated herein by reference), then it is preferred that the matrix be comprised of micron sized, or nano-sized particles, for example from about 0.1 nm to about 100 μm, or any size therebetween.

Non-limiting examples of a matrix that may be used according to the present invention include skim milk powder, soya protein powder, whey protein powder, albumin powder, casein, gelatin, plant peptone, algal protein and other single cell proteins, trehalose, mannitol or other powdered sugar or sugar alcohol, charcoal, or latex beads or other inert surfaces, water-soluble carbohydrate-based materials, talc, chitin, fish cartilage, and the like, or a combination thereof. Preferably, the matrix is generally regarded as safe (GRAS). In the present description, bacteriophages, or phages components that are non-covalently associated with the matrix (adsorbed), or covalently associated with a matrix, will be referred to as “immobilized phages” or “immobilized bacteriophages”.

The bacteriophages, or phage components, in aqueous solution may be applied to the matrix by any method known in the art, for example dripping or spraying, provided that the amount of the matrix exceeds the amount of aqueous bacteriophage, or phage components, solution. It is preferred that the matrix remain in a dry or semi-dry state, and that a liquid suspension of bacteriophages (or phage components) and matrix is not formed. Of these methods, spraying the bacteriophage solution over the matrix is preferred.

The antibacterial composition comprising bacteriophages, or phage components, and matrix may be dried at a temperature from about 0° C. to about 50° C. or any amount therebetween, for example at a temperature of 0, 2, 4, 6, 8, 10, 12, 14, 16, 18, 20, 22, 24, 26, 28, 30, 32, 34, 36, 38, 40, 42, 44, 46, 48, or 50° C. The antibacterial composition may be dried at a temperature from about 10° C. to about 30° C., or any amount therebetween, or from about 15° C. to about 25° C. or any amount therebetween. The drying process may also be accelerated by providing a flow of air over or through the antibacterial composition. Alternatively, the drying may be performed by heating the immobilized material under vacuum.

After a period of drying, additional aqueous solution may be applied to the matrix if desired, and the matrix re-dried. This process may be repeated as required to obtain the desired amount of phage on the matrix. The titer of phage on the matrix can be readily determined using standard techniques.

Alternatively, the antibacterial composition may comprise bacteriophages, or phage components that are chemically bonded, or covalently attached to a substrate, for example, but not wishing to be limiting, as generally described in WO 03/093462 (Hugh et al, incorporated herein by reference in its entirety). The substrates for chemical bonding of bacteriophages, or phage components, may include, but are not limited to polymers, nylon, plastics, microbeads, and biological substances. Preferably, the substrate be comprised of micron sized, or nano-sized particles, for example from about 0.1 nm to about 100 μm, or any size therebetween.

In yet another alternative, the antibacterial composition may comprise bacteriophages, or phage components, that have been lyophilized. Lyophilization may occur by any suitable method known in the art, and may be performed under conditions to optimize the viability of the bacteriophages, or phage components. For example, but not wishing to be limiting in any manner, the bacteriophages, or phage components, may be lyophilized in the presence of a stabilizing agent (Jepson and March, 2004, Vaccine, 22:2413-2419). Any suitable stabilizing agent known in the art to protect proteins or viruses and maintaining viability can be used. Of particular interest as stabilizing agents during lyophilization are trehalose and heat shock proteins. Lyophilization of bacteriophage or phage components can be carried out using any known lyophilization procedure, for example but not limited to methods disclosed in Clark and Geary (1973, Preservation of bacteriophages by freezing and freeze-drying, Cryobiology, 10, 351-360; Ackermann et al. 2004, Long term bacteriophage preservation, World Federation Culture Collections Newsletter, 38, 35 (which are both incorporated herein by reference).

In a further alternative, the bacteriophages or phage components adsorbed to a matrix, the bacteriophages or phage components chemically bonded to a substrate, or the bacteriophages or phage components that have been lyophilized may be embedded in a solid support. Additionally, an aqueous solution of bacteriophage may be embedded within a solid support and dried. Any suitable solid support known in the art to provide a delayed release may be used, for example, but not to be limiting in any manner, microbeads, cellulose-based material, carbohydrate-based material, shellac, polymers, methacrylates, sugars for example but not limited to manitol and sorbitol, soya protein, whey protein, algal protein and other single cell proteins, casein, gelatin, milk powder, hydroxypropylmethylcellulose phthalate (HPMCP), cellulose acetate phthalate (CAP), and hydroxypropylmethylcellulose Acetate Succinate (HPMCAS).

The antibacterial compositions described above, whether it be bacteriophage, or phage components adsorbed to a matrix; bacteriophage, or phage components chemically bonded to a substrate; or bacteriophages, or phages components that have been lyophilized; or any of the aforementioned antibacterial compositions that have been embedded in a solid support, are referred to herein as “stabilized bacteriophage, or phage components” or “stabilized phages, or phage components”. Preferably, “stabilized bacteriophage, or phage components” or “stabilized phages, or phage components” comprise bacteriophage, or phage components adsorbed to a matrix. These compositions may be refereed to as “matrix-stabilized bacteriophage or phage components”. Bacteriophages, or phage components adsorbed to a matrix and embedded in a solid support; bacteriophages may be referred to “embedded-stabilized bacteriophage or phage components”. Bacteriophages, or phage components embedded in a solid support; bacteriophages may be referred to “embedded bacteriophage or phage components”. While bacteriophage or phage components chemically bonded to a substrate may be referred to “covalent-stabilized bacteriophage or phage components”. The stabilized phages described herein, when introduced within a liquid environment, may release the bacteriophages or phages components, such that the bacteriophages or phages components would be free in solution. Alternatively, the covalently-stabilized phage compositions may be comprised of particulate matter of a size (for example micron sized, or nano-sized particles, from about 0.1 nm to about 100 μm, or any size therebetween) that would enable the bacteriophages or phages components to be essentially free in solution, and able to interact with a target host.

The stabilized bacteriophages, or phages components, described above may be formulated using any suitable method known in the art. For example, but not wishing to be limiting in any manner, the stabilized bacteriophages may be encapsulated, incorporated into a capsule, tablet, or a combination thereof.

By “encapsulated” or “coated”, it is meant that the antibacterial composition is coated with a substance that increases the phages' resistance to the physico-chemical stresses of its environment. The stabilized phages, or phage components, may be coated with any substance known in the art, by any suitable method known in the art, for example, but not limited to the method described in US publication 2003/0109025 (Durand et al., which is incorporated herein by reference) In this method, micro-drops of the coating substance, either as a hot melt or an organic solution, are injected into a chamber containing the component to be encapsulated, and rapidly cooled. Alternatively, a coating composition may be admixed with the one, OT more than one stabilized bacteriophage, or phage components, of the present invention, with constant stirring or agitation, and cooled or dried as required. The encapsulated composition may be reintroduced into the chamber in order to increase the thickness of the coating. In this manner, encapsulated bacteriophage compositions having different coating thickness may be obtained that exhibit varied time-released properties within a suitable environment.

In another alternative method of the present invention, stabilized bacteriophage are encapsulated or coated using spinning disk atomization (e.g. U.S. Pat. No. 5,643,594; U.S. Pat. No. 6,001,387; U.S. Pat. No. 5,578,314; Senuma Y et. al. 2000, Biomaterials 21:1135-1144; Senuma Y., at. al. 2000, Biotechnol Bioeng 67:616-622; which are incorporated herein by reference). As would be understood by a person of skill in the art, the stabilized phages may be dispersed in either a hot melt or an organic solution containing the desired coating substance, provided that the bacteriophage remain viable under the conditions, or at the temperature, selected. In the case where phage components are used, a higher hot melt temperature may be used. The dispersion may then be fed onto the center of a rotating disk and the material is atomized as it leaves the periphery of the disk, resulting in encapsulated stabilized bacteriophages. The encapsulated material may then be cooled or dried and collected using a cyclone separator, or a bed of modified food starch. The encapsulated composition may be reintroduced into the spinning disk atomizer in order to increase the thickness of the coating. In this manner, encapsulated bacteriophage compositions having different coating thickness may be obtained that exhibit varied time-released properties within a suitable environment.

Air-suspension coating is yet another example of an encapsulation method that may be used with the bacteriophage or phage components of the present invention. In this method, a fluid-bed spray coater is used to apply a uniform coating, either hot melt or organic solution, onto solid particles (e.g. Jones, D. 1994, Drug Development and Industrial Pharmacy 20:3175-3206; Hall et al. 1980, The Wurster Process, in “Controlled Release Technologies Methods, Theory, and Applications”, Kyonieus A. F. ed. Vol 2, pp. 133-154, CRC Press, Boca Raton Fla.; Deasy, P. B., 1988, Crit. Rev. Ther. Drug Carrier Syst. S(2):99-139; which are incorporated herein by reference). The antibacterial composition may be suspended by an air stream that is configured to induce a smooth, cyclic-flow past a nozzle used to atomize the coating substance. Once sprayed, the antibacterial composition particles may be lifted by the air stream as the coating cools or dries. The particles may then be circulated past the nozzle until a uniform coating is obtained, or until the desired film thickness has been applied. In this manner, encapsulated bacteriophage compositions having different coating thickness may be obtained that exhibit varied time-released properties within a suitable environment.

Additional coating methods include but are not limited to fluid bed systems for dry granulation and coating, admixing with a solvent-coating substance mixture followed solvent evaporation, drying or both.

The coating substance may be any suitable coating substance known in the art. For example, but without wishing to be limiting, the coating substance may comprise a substance with a melting temperature (i.e. hot melt coating) between about 20° C. and about 100° C., for example between about 30° C. and about 80° C., or between about 60° C. and about 80° C., or any temperature therebetween; for example, the melting temperature may be 20, 22, 24, 26, 28, 30, 32, 34, 36, 38, 40, 42, 44, 46, 48, 50, 52, 54, 56, 58, 60, 62, 64, 66, 68, 70 72, 74, 76, 78, 80, 82, 84, 86, 88, 90, 92, 94, 96, 98, or 100° C., or any temperature therebetween, provided that the bacteriophage remain viable under the temperature selected. In the case where phage components are used, a higher hot melt temperature may be used. Alternatively, an organic solvent comprising the coating compound or substance may be used. Non-limiting examples of organic solvents include methylene chloride, methyl acetate, ethyl acetate, methyl ether ketone, acetone, alcohols and other solvents or combinations thereof.

If the coating substance is to be ingested or used for an oral application, then it is preferred that the coating substance is a food grade substance. However, the bacteriophage, or phage component, composition of the present invention may also be coated with other substances that are not food grade, depending on the antibacterial composition's intended use. For example, the antibacterial composition may be encapsulated in an emulsion-compatible coating for use in lotions or lubricants or creams or gels. Other additive molecules may be added to the coating substance; such additive may include antioxidants, sugars, proteins or other synthetic material.

Non-limiting examples of suitable coating substances include lipid-based materials such as vegetable fatty acids; fatty acids such as palmitic acid and stearic acid, for example Stéarine™, animal waxes for example beeswax, vegetable waxes, for example Carnauba wax or Candelilla wax, wax derivatives, other lipids or lipid derivatives, and shellac.

Additional coating substances may also be used to encapsulate the bacteriophage, phage components, or both, of the present invention. For example, non lipid-based materials (see for example, U.S. Pat. Nos. 6,723,358; and 4,230,687, both of which are incorporated herein by reference), including but not limited to sugars, cellulose-based components, or other carbohydrate-based components that may be water soluble may be used. Additional examples of non-lipid based materials suitable for encapsulation include, without wishing to be limiting in any manner, hydroxypropylmethylcellulose phthalate (HPMCP), cellulose acetate phthalate (CAP), or hydroxypropylmethylcellulose Acetate Succinate (HPMCAS).

Additionally, enteric film coatings may used to coat the stabilized bacteriophage of the present invention, for example, but not limited to anionic polymers of methacrylate acid and methacrylates (containing —COOH as a functional group) and dissolving at pH 5.5 to about 7.0, for example EUDRAGIT™ related coating including aqueous dispersions for targeting the duodenum or colon (e.g. EUDRAGIT™ L30 D55, EUDRAGIT™ FS 30D), solid substances for targeting the duodenum, jejunum, or ileum (EUDRAGITL™ 100-55, EUDRAGIT™ L100, EUDRAGIT™ S100), organic solvents for targeting the jejunum, or ileum (EUDRAGIT™ L12,5, EUDRAGIT™ 12,5). Examples of a sustained release polymers include coating compositions comprising copolymers of acrylate and methacrylates with quartenary ammonium groups as functional groups, and ethacrylate methacrylate copolymers with a neutral ester group, and mixtures thereof, for example EUDRAGIT™ RL (permeable composition), EUDRAGIT™ RS (a poorly permeable composition), and EUDRAGIT™ NE (swellable and permeable composition).

Polymers may also be used to encapsulate or coat the stabilized bacteriophages or phage components. Any polymer known in the art may be used, and may be chosen for immediate release of the bacteriophages, or phage components, or for controlled release. For example, and without wishing to be limiting in any manner, appropriate polymers may include those disclosed by Mehta (U.S. Pat. No. 6,926,909), Hussain et al (U.S. Pat. No. 6,649,187), or Yang et al (U.S. Pat. No. 5,576,022), all of which are incorporated herein by reference. In a non-limiting example, polymers such as poly(vinyl acetate) phthalate (PVAP), methacrylates, or shellac may be used.

As would be understood by a person of skill in the art, one or more than one of the coating substances may be used. For example, the stabilized bacteriophages, or phage components, may be encapsulated using one type of coating substance, followed by a second encapsulation, or over-coating, using another coating substance. In a non-limiting example, the stabilized bacteriophages, or phage components may first be encapsulated with or embedded in a cellulose-based component, followed by over-coating with a lipid-based material; alternatively, the stabilized bacteriophages, or phage components may first be encapsulated with or embedded in a polymer, followed by over-coating with a lipid-based, or other water-resistant material.

The process of lipid-based encapsulation protects the bacteriophages, phage components, or both, to some extent from a harsh environment the bacteriophages or components may be exposed to, for example, the low pH environment over a range of fermenting liquid feed conditions, or the digestive system of an animal. The lipid-based material selected for encapsulation should also exhibit the property that it breaks down within a desired environment so that the bacteriophages or phage components are released, providing one form of timed-release. For example, digestive enzymes may degrade the encapsulating material and assist in the release of the bacteriophages or phage components within the gut of an animal, or enzymes within the fermenting liquid feed, for example, may assist in the release of some of the bacteriophages or phage components from encapsulation. Other mechanisms of release include pH-based, and reaction with chemicals released within a defined chamber such as bile acids. As a result, several materials for encapsulating the bacteriophages or phage components may be used so that if desired, there is selected release of the bacteriophage, while at the same time protecting the bacteriophages, or phage components. Varying the thickness of the coating of encapsulated bacteriophages or phage components may also provide additional timed-release characteristics.

In addition, a non-lipid-based or polymer encapsulation material may dissolve in water, thereby releasing bacteriophages or phage components immediately, or soon after mixing with the liquid feed medium. The bacteriophages or phage components may also be released in a time-controlled fashion depending upon the material and formulation selected, or whether the preparations are provided within a capsule or tablet form. The capsule or tablet formulations may assist in the timed release of the stabilized bacteriophages or phage components within the animal or other environment. Therefore, mixtures of controlled release bacteriophages, phage components, or both that are admixed and/or encapsulated with different materials may be combined and mixed with animal feed, liquid animal feed, or otherwise administered to an animal.

As would be understood by a person of skill in the art, the encapsulated formulations may comprise stabilized bacteriophage, stabilized phage components, lyophilized bacteriophage, lyophilized phage components, or a combination thereof, in on or more than one form. For example, the encapsulated product may comprise:

bacteriophage, phage components, or a combination thereof, adsorbed to a matrix and encapsulated

bacteriophage, phage components, or a combination thereof, adsorbed to a matrix, embedded in a solid support, and encapsulated;

bacteriophage, phage components, or a combination thereof, embedded in a solid support, and encapsulated

bacteriophage, phage components, or a combination thereof, chemically bonded or covalently linked to a substrate and encapsulated;

bacteriophage, phage components, or a combination thereof, chemically bonded or covalently linked to a substrate, embedded in a solid support, and encapsulated;

bacteriophage, phage components, or a combination thereof, that have been lyophilized and encapsulated;

bacteriophage, phage components, or a combination thereof, that have been lyophilized, embedded in a solid support, and encapsulated,

or mixtures thereof.

The stabilized bacteriophage, phage components, or a combination thereof, or the encapsulated bacteriophage, phage components, or a combination thereof, may also be provided in a capsule form. By “capsule form”, it is meant that the stabilized bacteriophage, encapsulated phage, stabilized or encapsulated phage components, or a combination thereof, are provided in a capsule form, for example, a soft capsule suitable for pharmaceutical use, which may be solubilized within an aqueous environment. The capsule may be made of any suitable substance known in the art, for example, but not limited to gelatin, shellac, methacrylates, a synthetic polymer, wax or other compounds, and may comprise additional components such as stabilizers and colorants, as would be known to a person of skill in the art.

The stabilized bacteriophage or phage components, encapsulated bacteriophage or phage components, or a combination thereof, may also be provided in a tablet form. By “tablet form”, it is meant that the stabilized phages, or phage components, are provided in a pressed tablet that dissolves in an aqueous environment. The tablet may be made of any suitable substance known in the art, by any suitable method known in the art and may be comprised of pharmaceutically acceptable ingredients. For example, the tablet may comprise binders and other components necessary in the production of a tablet as are known to one of skill in the art. The tablet may be an immediate release tablet, where the bacteriophages or phage components are released into the liquid feed upon dissolution of the tablet, or may comprise a timed-release composition, where the bacteriophages or phage components are released within an aqueous environment in a time-dependent manner. See WO 02/45695; WO 03/051333; U.S. Pat. No. 4,601,894; U.S. Pat. No. 4,687,757, U.S. Pat. No. 4,680,323, U.S. Pat. No. 4,994,276, U.S. Pat. No. 3,538,214, US (which are incorporated herein by reference) for several examples of time-release formulations that may be used to assist in the time controlled release of bacteriophages, or phage components within aqueous environments.

Tablet formulations may comprise a hydrodynamic fluid-imbibing polymer for example but not limited to acrylic-acid polymers with cross-linking derived from allylsucrose or allylpentaerithritol, including water-insoluble acrylic polymer resins. Single compounds or a blend of compounds from this group of polymers include for example, but not limited to Carbopol®.971-P, Carbopol®.934-P, Carbopol®.974P and Carbopol®.EX-507 (GF Goodrich, or any other commercially available brand with similar properties, may be used). Preferably, the acrylic-acid polymers have a viscosity from about 3,000 centipoise to about 45,000 centipoise at 0.5% w/w concentration in water at 25EC, and a primary particle size range from about 3.00 to about 10.00 microns in diameter, as determined by Coulter Counter; highly cross-linked or lightly cross-linked starch derivatives crosslinked by Epichlorhydrin or Phosphorous oxychloride (POCl₃) or Sodium trimetaphosphate either singly or in blends; polyglucans such as amylose, dextran, pullulan succinates and glutarates containing diester—cros slinks either singly or in blends; diether crosslinked polyglucans such as those disclosed in U.S. Pat. Nos. 3,208,994 and 3,042,667 (which are incorporated herein by reference); crosslinked polyacrylate resins such as, but not limited to, potassium polyacrylate; and water swellable crosslinked polymer compositions of crosslinked polyethylenimine and or crosslinked polyallyamine.

Methods of preparation, for example of Carbopol®.934-P,—a polymer of acrylic acid lightly cross-linked with polyallyl ether of sucrose having an average of 5.8 allyl groups per each sucrose molecule, may be found in U.S. Pat. Nos. 2,909,462; 3,033,754; 3,330,729; 3,458,622; 3,459,850; and 4,248,857 (which are incorporated herein by reference). When Carbopol®.971-P is used, the preferred viscosity of a 0.5% w/w aqueous solution is 2,000 centipoise to 10,000 centipoise. More preferably, the viscosity of a 0.5% w/w aqueous solution is 3,000 centipoise to 8,000 centipoise. When Carbopol®.934-P is used, the preferred viscosity of a 0.5% w/w aqueous solution is 20,000 centipoise to 60,000 centipoise, more preferably, the viscosity of a 0.5% w/w aqueous solution is 30,000 centipoise to 45,000 centipoise

Cross-linked starch derivatives (crosslinked by Epichlorhydrin or Phosphorous oxychloride (POCl.sub.3) or Sodium trimetaphosphate) include high amylose starch containing varying degrees of crosslinking. These compounds and their methods of preparation are known in the art, for example, U.S. Pat. No. 5,807,575 and U.S. Pat. No. 5,456,921 (which are incorporated herein by reference), and Rutenberg and Solarek (M. W. Rutenberg and D. Solarek, “Starch derivatives: production and uses” in Starch Chemistry and Technology, 2^{nd} Edition, Chapter X, Pages 311-379, R. L. Whistler, J. N. BeMiller and E. F. Paschall, Academic Press, 1984; which is incorporated herein by reference).

Tablet formulations may be formulated to comprise an agent that expands rapidly upon exposure to fluid, for example, a rapid expansion polymer. For example, this agent may comprise hydrophilic cross-linked polymers that are capable of rapid capillary uptake of water and a limiting volume expansion. Non-limiting examples of rapid expansion polymers include: single compounds or combinations derived from cross-linked N-vinyl-2-pyrollidone (PVP) selected from a group of chemically identical polyvinylpolypyrrolidone such as Polyplasdone®.XL, Polyplasdone®.XL-10, Polyplasdone®.INF-10 (International Specialty Products). Preferably, the cross-linked N-vinyl-2-pyrollidone has a particle size from about 9 microns to about 150 microns; and cross-linked cellulose derivatives selected from a group of hydrophilic compounds such as cross-linked carboxymethyl cellulose (for example croscarmellose), sodium starch glycolate or a combination thereof.

As would be understood by a person of skill in the art, the capsule or tablet formulations may comprise stabilized bacteriophage optionally including phage components, encapsulated bacteriophage optionally including phage components, or a combination thereof, in on or more than one form. For example, the capsule or tablet may comprise:

bacteriophage, phage components, or a combination thereof, adsorbed to a matrix;

bacteriophages, or phage components adsorbed to a matrix and encapsulated;

bacteriophage, phage components, or a combination thereof, adsorbed to a matrix and embedded in a solid support;

bacteriophage, phage components, or a combination thereof, adsorbed to a matrix, embedded in a solid support, and encapsulated;

bacteriophage, phage components, or a combination thereof and embedded in a solid support;

bacteriophage, phage components, or a combination thereof, embedded in a solid support, and encapsulated;

bacteriophage, phage components, or a combination thereof, chemically bonded or covalently linked, to a substrate;

bacteriophage, phage components, or a combination thereof, chemically bonded or covalently linked to a substrate and encapsulated;

bacteriophage, phage components, or a combination thereof, chemically bonded or covalently linked to a substrate and embedded in a solid support;

bacteriophage, phage components, or a combination thereof, chemically bonded or covalently linked to a substrate, embedded in a solid support, and encapsulated;

bacteriophage, phage components, or a combination thereof, that have been lyophilized;

bacteriophage, phage components, or a combination thereof, that have been lyophilized and encapsulated;

bacteriophage, phage components, or a combination thereof, that have been lyophilized and embedded in a solid support;

bacteriophage, phage components, or a combination thereof, that have been lyophilized, embedded in a solid support, and encapsulated,

or mixtures thereof.

By “controlled release” or “timed release”, it is meant that the agent administered to the animal is released from the formulation in a time-dependent manner. For example, the one or more than one bacteriophage or phage component may be stabilized bacteriophage, encapsulated bacteriophage, stabilized phage components, encapsulated phage components, bacteriophage or phage components that are provided in capsule form, bacteriophages or phage components that are provided in tablet form, bacteriophages or phage components that are encapsulated, in capsules, in tablets, or a combination thereof, wherein the encapsulated, capsule, or tablet forms of the bacteriophages or phage components comprise compositions that release the bacteriophages or phage components at different rates within the appropriate environment, for example an aqueous environment. The compositions of the encapsulation material, capsule, or tablets may include polymers, waxes, gels, compounds that imbibe water, repel water, or both, fatty acids, sugars, proteins or synthetic materials, to effect release of an agent within the composition in a controlled manner. Various controlled release compositions comprising bacteriophages or phage components may be used so that the bacteriophages or phage components may be released at different times with the appropriate environment, for example, within a liquid feed composition, prior to administration to an animal, during passage through the digestive tract of the animal, or after leaving the animal.

The antibacterial compositions of the present invention exhibit desirable storage properties and may be used in a variety of applications. For example, which is not to be considered limiting in any manner, the antibacterial compositions may be used for human, veterinary, aquacultural, and agricultural applications. For example, encapsulated bacteriophage may be admixed with fish feed for use within aquaculture applications, including farming and maintenance of fish for food and fish for decorative purposes, such as tropical fish. Furthermore, the compositions may be used for the treatment of trees and plants, and environmental applications. For example, the antibacterial composition may be mixed with the feed of livestock, birds, poultry, domestic animals and fish, to aid in reducing the shedding of target bacteria. Encapsulated phages may be mixed with other additives or supplements applied to animal feed as part of the daily feed regime, as needed. Thus, settling of the bacteriophages, or phage components, in the feed could be avoided. Alternatively, the adhesion of the feed or the encapsulated phage, or both, may be enhanced to provide improved mixing and delivery. In another example, the antibacterial material, alone or in combination with a pharmaceutically acceptable carrier or excipient that will not affect the activity or specificity of the bacteriophages, or phage components, could be used as an oral, medication for humans, mammals, or avian species. The encapsulated bacteriophage may also be used within phage therapy applications including human, veterinary, agricultural applications. Furthermore, encapsulated bacteriophage may be admixed with fish feed for use within aquaculture applications, including farming and maintenance of fish for food and fish for decorative purposes, such as tropical fish.

Therefore, the present invention provides an antibacterial composition comprising one or more than one strain of bacteriophage, one or more than one phage component, or a combination thereof, adsorbed onto a matrix, or dispersed within a pharmaceutically acceptable carrier, a cream, lotion, lubricant, gel, or a combination thereof. The present invention also provides an antibacterial composition comprising one or more than one strain of bacteriophage, one or more than one phage component, or a combination thereof, adsorbed onto a matrix, encapsulated or present within a time-release formulation. The encapsulated, or time-release bacteriophage formulation may be dispersed within a pharmaceutically acceptable carrier, a cream, lotion, lubricant, gel, or a combination thereof.

The present invention also provides a kit comprising an antibacterial composition, the antibacterial composition comprising one or more than one strain of bacteriophage, one or more than one phage component, or a combination thereof, adsorbed onto a matrix, and a vial of sterile water or media for dissolving the composition. The present invention further provides a kit comprising an antibacterial composition, the antibacterial composition comprising one or more than one strain of bacteriophage, one or more than one phage component, or a combination thereof, adsorbed onto a matrix and encapsulated or within a time-release formulation, and a vial of sterile water or media for dissolving the composition.

The present invention also provides a method of treating a wound or a skin infection comprising, applying an antibacterial composition as described herein, for example an encapsulated antibacterial composition, or a time-release antibacterial composition, comprising one or more than one strain of bacteriophage, one or more than one phage component, or a combination thereof, a pharmaceutically acceptable carrier, a cream, lotion, lubricant, gel, or a combination thereof, to the wound, or skin infection. Furthermore, the antibacterial composition of the present invention may be used to treat a bacterial infection within an animal. Such treatment may involve introducing the antibacterial composition to the animal nasally or orally, for example the composition may be administered as a powder inhalant, or as an additive in feed.

The present invention also provides a composition comprising an animal feed admixed with an antibacterial composition as described herein, for example an antibacterial composition that has been encapsulated, a time-release antibacterial composition, comprising one or more than one strain of bacteriophage, one or more than one phage component, or a combination thereof, where the composition comprises one or more than one strain of bacteriophage. The animal feed may be selected from the group consisting of a bird feed, a fish feed, a porcine feed, a livestock feed, a poultry feed, a domestic animal feed, and a food for aquaculture.

The present invention will be further illustrated in the following examples.

### EXAMPLES

### Example 1

### Isolation, Amplification and Titration of Phage

Bacteriophages were isolated from manure samples obtained from dairy and beef farms across Canada. Manure samples were allowed to react with E. coli O157:H7 and plated onto agar plates. Any phage plaques obtained were isolated and purified as per standard phage purification protocols (Maniatis et al (1981) Molecular cloning: a laboratory manual, Cold Spring Harbor Laboratory, Cold Spring Harbor, N.Y.).

Purified phages isolated as outlined in Example 1 were amplified using the isolation strain of E. coli O157:H7. Purified phage and bacteria were mixed together, let stand at room temperature for 10 minutes, and amplified according to standard protocols commonly used in the art (Maniatis et al (1981) Molecular cloning: a laboratory manual, Cold Spring Harbor Laboratory, Cold Spring Harbor, N.Y.). Amplified samples in LB broth were filter sterilized and used.

Concentrations of bacteriophage solutions were determined using standard phage titration protocols (Maniatis et al (1981) Molecular cloning: a laboratory manual, Cold Spring Harbor Laboratory, Cold Spring Harbor, N.Y.). Preparations containing phages were diluted with LB, mixed and incubated with E. Coli O157:H7 for 10 minutes and plated onto agar plates. The concentration of phages was determined from the number of plaques obtained at the different dilutions and multiplying with the appropriate dilution factor.

### Example 2

### Immobilization of Phages

E. coli O157:H7 specific phages P10 and R4, prepared as described in example 1, were immobilized on two different matrices: powdered milk (fat free) and soya protein. Both milk powder (Carnation) and soya protein (Supro) were obtained off-the-shelf from local food stores. Identical protocols were used for both materials.

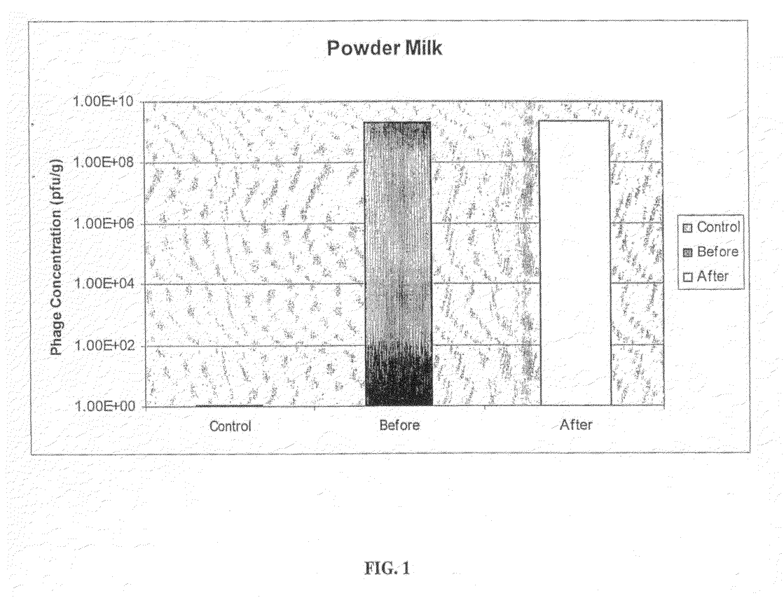

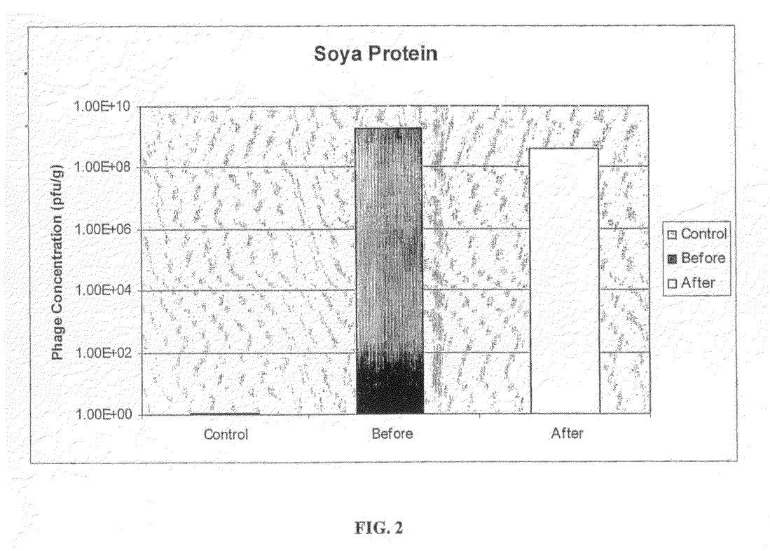

50 g of powder (powdered milk or soya protein) was spread in a glass dish. Phages in solution were uniformly sprayed onto each powdered matrix. Varying titers of phages, ranging from 10⁵ pfu/g to 10⁹ pfu/g, were-used with powdered milk, each yielding similar results. The phage-powder was mixed and dried at 37° C. for 2 hours, or until completely dried. The resulting bacteriophage composition was ground into a fine powder, with particle sizes in the range of 50-600 μm and an average particle size of 200 μm. 0.5 grams of each powdered bacteriophage composition was re-suspended in 10 ml of reverse-osmosis (RO) water and the recovery of phages tested. Powdered milk or powdered soya protein in the absence of bacteriophages was used as a control. Slight clumping of the bacteriophage composition comprising soy protein was observed when re-suspended in the RO water. The results for bacteriophage compositions prepared using dry milk power as the matrix are presented in FIG. 1. Results for bacteriophage compositions prepared using soy protein as the matrix are presented in FIG. 2.

For phage immobilized on powdered milk, the results show that phage can be recovered from the bacteriophage composition and no loss in activity was observed. FIG. 1 shows that the phage titer obtained after immobilization (“After”) is similar to the amount of phage added to the powder (“Before”). Similar results were observed for bacteriophage compositions comprising soy protein (FIG. 2; After—immobilized phage; Before—amount of phage added to matrix). For phage immobilized on soya protein, a slight decrease in phage recovery was observed which may be due to caking of the soya protein upon addition to RO water.

These results also show that immobilized phages are readily released from a matrix when introduced to an aqueous medium.

### Example 3

### Encapsulation of Bacteriophage Compositions

Bacteriophage compositions are prepared as described in Example 2, and are provided as microcapsules or microspheres. Generally, microcapsules are produced by coating the bacteriophage composition in a solid support including, soya protein, or milk powder (other solid supports include microbeads, cellulose-based material, carbohydrate-based material, manitol, sorbitol, whey protein, algal protein, single cell protein, casein, gelatin, shellac) with a particle size of approximately 1 mm. Microspheres are produced by grinding the stabilized phage preparation and mixing it in lipid melt to produce microspheres of from about 0.01 mm to about 1 mm.

The microcapsules are then encapsulated by either spinning-disk atomization, air-suspension coating using a fluid-bed spray coater, or Bacteriophage using the method disclosed in US 2003/0109025 (which is incorporated herein by reference in its entirety).

Briefly, for spinning-disk atomization, phage compositions are dispersed in either a hot melt, between about 30° C. and about 80° C., or an organic solution including methylene chloride, methyl acetate, ethyl acetate, methyl ether ketone, acetone, or alcohol, and containing the coating substance. The dispersion is fed onto the center of a rotating disk and the material is atomized as it leaves the periphery of the disk. The encapsulated material is collected using a cyclone separator or a bed of modified food starch.

In air-suspension coating, a fluid-bed spray coater is used. Solid particles are suspended by an air stream that induces a smooth, cyclic-flow past a nozzle that atomizes the coating substance. Once sprayed, the particles are lifted by the air stream as the coating dries. The particles are circulated past the nozzle until a uniform coating is applied. The particles are circulated until the desired film thickness has been applied. Microspheres are encapsulated using spinning-disk atomization.

Each of the microcapsules and microspheres are encapsulated with each of the following coating substances: palmitic/stearic acid (e.g. Stëarine 50/50, obtained from Exaflor, Gif sur Yvette, France), shellac over-coated with palmitic acid, stearic acid, HPMCP over-coated with palmitic acid, stearic acid, CAP over-coated with palmitic acid, stearic acid, HPMCAS over-coated with palmitic acid, stearic acid, PVAP over-coated with palmitic acid, stearic acid, and methacrylate over-coated with palmitic acid, stearic acid. Other fatty acids may also be used in a similar manner for over-coating.

Once the coating operation is complete, the encapsulated stabilized phage particles are collected and stored in airtight containers.

The effect of encapsulation on the titer of bacteriophage compositions is evaluated by determining the activity of the stabilized phage preparation before (“Before”) and after (“After”) encapsulation. For this analysis, encapsulated bacteriophages are re-suspended, and ground using a blender. The re-suspended encapsulated stabilized bacteriophages are blended or treated with an appropriate release mechanism, including exposure to an aqueous solution, a pH change, enzymatic digestion, or a combination thereof, in order to disrupt the encapsulated particles and release the bacteriophages. For bacteriophage release by blending, 0.5 g of encapsulated stabilized phages are mixed with 45.5 ml of re-suspension media (LB Broth or RO Water), and 250 μl of antifoam agent is added to prevent foaming upon grinding.

The results demonstrate that bacteriophages can be recovered from an encapsulated bacteriophage composition, and encapsulation does not inactivate the immobilized phage.

### Example 4

### Stability and Release of Encapsulated Bacteriophages

Phages are encapsulated as described in Example 3. The release of encapsulated stabilized phages upon physical or chemical disruption is tested in the following manner: 0.5 g of encapsulated stabilized phage is mixed with 45.5 ml of re-suspension media (LB Broth or RO Water). 250 μl of antifoam agent is used to prevent foaming upon grinding. A control sample of encapsulated stabilized phages is prepared as described above, but not subjected to grinding, to determine the non-specific leaching of encapsulated bacteriophages within the re-suspension medium.

The stability of the encapsulated bacteriophages at low pH is also examined. After re-suspension (as outlined above), the encapsulated stabilized phages are incubated for 30 or 60 min at pH 2.15, neutralized to pH 7.0 using NaOH, then ground using a blender; another sample (control) is resuspended and immediately ground. Both the control and test samples are filter sterilized using a 0.45 μm syringe filter prior to use.

The results demonstrate that bacteriophages may be released following disruption of encapsulated bacteriophage particles. Furthermore, these results shows that encapsulated bacteriophage may be exposed to a pH of 2.15 for prolonged period of time, with little or no loss in activity (titer). The results for non-encapsulated and non-stabilized bacteriophages are consistent with the results of Jepson and March (2004, Vaccine, 22:2413-2419), where a dramatic loss of viability of bacteriophages was observed after only 5 minutes at pH below pH 2.2. This loss in activity is obviated by encapsulation of the bacteriophages as described in the present invention.

### Example 5

### Stability of Immobilized Phage

Bacteriophages were immobilized on a matrix, in this case milk powder as described in Example 2 and the material was stored at either room temperature (RT) or at 4° C. (4 C) in airtight containers. Samples were obtained at different time points, and phage titers determined, over a period of 10 months. The initial phage concentration was 3×10⁶ pfu/g.

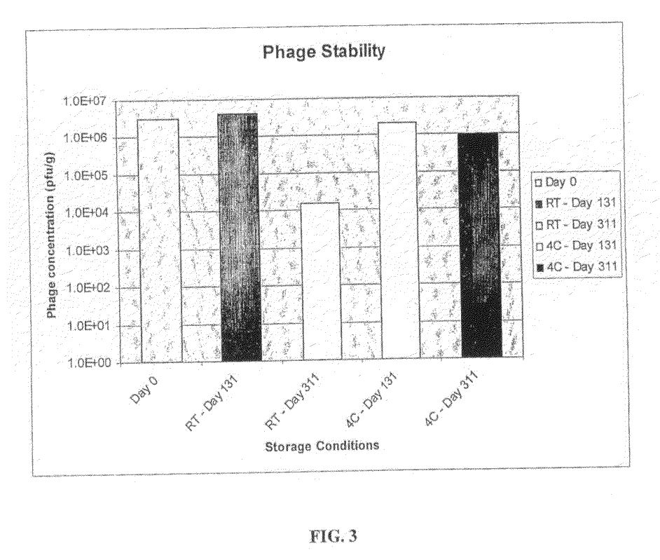

FIG. 3 shows that the immobilized phages (bacteriophage composition) are stable at either room temperature or 4° C. for at least 131 days (4.5 months), and is stable for at least 311 days (10 months) at 4° C. Addition of a desiccant, or storage of the bacteriophages in a desiccated environment may further increase the viability of the bacteriophage composition.

### Example 6

### Immobilized Phage in Cream and Lotion

The viability of immobilized phages (bacteriophage composition) incorporated into a lotion or cream was also investigated.

Two grams of lotion (Vaseline hand lotion) or cream (GlycoMed cream) was weighed into a sterile Petri dish. The desired pfu/g of immobilized phage, P10 and R26, was added to the lotion or cream and mixed thoroughly. Bacteria were spread on LB-agar plates and allowed to grow at 37° C. for two to three hours to form a uniform lawn. Two cm² pieces of filter paper, two per plate, were placed onto the lawn and the lotion comprising bacteriophages, or the cream comprising bacteriophages, were each spread over one filter paper. Aliquots of the lotion or cream without phage (control) were spread onto the other filter paper to determine whether the lotion or cream had antimicrobial properties. A spot of lotion or cream containing bacteriophages was also placed directly on the bacterial lawn. Several dilutions of the bacteriophages within each of the lotion or cream were tested. The plates were incubated overnight at 37° C. Each treatment was scored as a “Yes” or a “No”, depending on the presence or absence of the zone of inhibition, respectively, and the results are presented in Table 1.

TABLE 1
Efficacy of bacteriophage compositions (immobilized
phages) in hand lotion or cream.

| Mate- | Tech- | | pfu/g | | | |
|---|---|---|---|---|---|---|
| rial | nique | Phage | 1.00E+07 | 1.00E+06 | 1.00E+05 | 1.00E+04 |
| Lotion | Filter | P10 | Yes | Yes | Yes | Yes (5/6) |
| Lotion | Spot | P10 | Yes | Yes | Yes | Yes (1/3) |
| Lotion | Filter | — | No | No | No | No |
| Cream | Filter | P10 | Yes | Yes | Yes | Yes |
| Cream | Spot | P10 | Yes | Yes | Yes | Yes |
| Cream | Filter | — | No | No | No | No |
| Lotion | Filter | R26 | Yes | Yes | Yes (2/3) | Yes (1/3) |
| Lotion | Spot | R26 | Yes | Yes | Yes | No |
| Lotion | Filter | — | No | No | No | No |
| Cream | Filter | R26 | Yes | Yes | Yes | Yes |
| Cream | Spot | R26 | Yes | Yes | Yes | Yes |
| Cream | Filter | — | No | No | No | No |

A zone of inhibition of bacterial growth was observed where activity of phages could be recovered. Lotion and cream containing encapsulated immobilized phages both show antibacterial activity, while the lotion or cream alone shows no inhibition of bacterial growth. These results indicate that bacteriophage compositions prepared according to the present invention may be admixed within lotion and cream preparations for use as antibacterial lotions or creams.

Improved stability of the bacteriophages is observed for immobilized bacteriophages in creams.

### Example 7

### Delivery of Active Bacteriophages

E. coli 0157 specific bacteriophages are encapsulated as previously described in Examples 3 and 4. The encapsulated phages are then mixed with other supplements and added to animal feed in an amount of about 1-50 g per animal per dose. The feed is then fed to the animal once per day for 5 to 7 days prior to slaughter. Alternatively, a maintenance dose is given to the animal every 1-3 days.

Analysis of the animal's manure reveals a decrease in the E. coli 0157 in the animal, indicating that active bacteriophages are delivered to the gut of the animal.

All citations are hereby incorporated by reference.

The present invention has been described with regard to one or more embodiments. However, it will be apparent to persons skilled in the art that a number of variations and modifications can be made without departing from the scope of the invention as defined in the claims.

The embodiments of the invention in which an exclusive property of privilege is claimed are defined as follows:

1. A method for producing bacteriophage composition comprising:
a) providing stabilized bacteriophage, phage components, or a combination thereof; and
b) encapsulating the stabilized bacteriophage, phage components, or a combination thereof, to produce the bacteriophage composition.

2. The method of claim 1, wherein the bacteriophage composition is encapsulated using a material selected from the group consisting of vegetable fatty acids, fatty acid, stearic acid, palmitic acid, an animal wax, beeswax, a vegetable wax, carnauba wax, candelilla wax, a wax derivative, a polymer, a cellulose-based material, hydroxypropylmethylcellulose phthalate, cellulose acetate phthalate, hydroxypropylmethylcellulose acetate succinate, a carbohydrate-based material, shellac, and a sugar.

3. The method of claim 2, wherein the step of encapsulation (step b)) is carried out using spinning disk atomization, air suspension coating, fluid bed system, solvent evaporation, or coating using solution comprising a solvent and coating compound.

4. The method of claim 1, wherein in the step of providing (step a)), the stabilized bacteriophage is stabilized by adsorption to a matrix.

5. The method of claim 4, wherein the matrix is selected from the group consisting of skim milk powder, soya protein powder, whey protein powder, albumin powder, casein, gelatin, single cell protein, algal protein, plant peptone, trehalose, mannitol, powdered sugar, sugar alcohol, charcoal, latex beads, a water-soluble carbohydrate-based material, talc, chitin, and fish cartilage.

6. The method of claim 1, wherein after the step of encapsulating (step b)), the bacteriophage composition is formulated as a capsule or a tablet.

7. The method of claim 1, wherein the step of providing (step a)), the stabilized bacteriophage is stabilized by adsorption to a matrix, stabilized by adsorption to a matrix and the matrix embedded in a solid support; lyophilized; lyophilized and embedded in a solid support, covalently bound to a matrix, covalently bound to a matrix and embedded in a solid support

8. The method of claim 7, wherein the solid support is selected from the group consisting of a microbead, cellulose-based material, carbohydrate-based material, shellac, polymers. methacrylates, sugar, manitol, sorbitol, soya protein, whey protein, algal protein, single cell protein, casein, gelatin, and milk powder.

9. A bacteriophage composition comprising one or more than one strain of an encapsulated stabilized bacteriophage, one or more than one encapsulated phage component, one or more than one strain of a stabilized bacteriophage and one or more than one phage component encapsulated together, or a combination thereof.

10. The bacteriophage composition of claim 9, wherein the one or more than one phage component is selected from the group consisting of a phage tail, a phage protein, and a combination thereof.

11. The bacteriophage composition of claim 10, wherein the stabilized bacteriophage is stabilized by adsorption to a matrix.

12. The bacteriophage composition of claim 11, wherein the matrix is selected from the group consisting of skim milk powder, soya protein powder, whey protein powder, albumin powder, casein, gelatin, single cell protein, trehalose, mannitol, powdered sugar, sugar alcohol, charcoal, latex beads, carbohydrate-based material, talc, chitin, and fish cartilage.

13. The bacteriophage composition of claim 9, wherein the stabilized bacteriophage, phage components, or a combination thereof, is encapsulated using a material selected from the group consisting of vegetable fatty acids, fatty acid, stearic acid, palmitic acid, an animal wax, beeswax, a vegetable wax, carnauba wax, candelilla wax, a wax derivative, a polymer, a cellulose-based material, hydroxypropylmethylcellulose phthalate, cellulose acetate phthalate, hydroxypropylmethylcellulose acetate succinate, a carbohydrate-based material, shellac, methacrylates, methacrylic acid, and a sugar.

14. The bacteriophage composition of claim 13, further comprising a pharmaceutically acceptable carrier.

15. The bacteriophage composition of claim 14, wherein the bacteriophage composition is formulated as a capsule or a tablet.

16. A method of improving bacteriophage stability comprising encapsulating stabilized bacteriophages or phage components, according to claim 1, and storing the encapsulated stabilized bacteriophages.

17. A composition comprising an animal feed admixed with the bacteriophage composition of claim 9.

18. The composition of claim 17, wherein the animal feed is selected from the group consisting of human feed, a bird feed, a fish feed, a porcine feed, a livestock feed, a poultry feed, a domestic animal feed, and a food for aquaculture.

19. A method for producing an antibacterial composition comprising, embedding an aqueous solution of bacteriophages, phage components, or a combination thereof onto a solid or powdered support to produce a composition, and drying the composition to produce an antibacterial composition.

20. The method according to claim 19, wherein the support may be selected from the group consisting of skim milk powder, soya protein, whey protein, albumin powder, casein, gelatin, single cell proteins, trehalose, manitol, sugar and sugar alcohol, talc, chitin, fish cartilage, hydroxypropylmethylcellulose phthalate (HPMCP), cellulose acetate phthalate (CAP), or hydroxypropylmethylcellulose Acetate Succinate (HPMCAS), and the like.

21. The method of claim 19, wherein the antibacterial composition is encapsulated.

22. The method of claim 21, wherein material used to encapsulate the antibacterial composition is selected from the group consisting of vegetable fatty acid, fatty acid, stearic acid, palmitic acid, an animal wax, a vegetable wax, Carnauba wax and other wax derivatives thereof, other lipids and lipid derivatives, shellac, a polymer, a cellulose-based material, a carbohydrate-based material, a methacrylate, methacrylic acid, or a sugar.
